(19) United States
(12) Patent Application Publication
(10) Pub. No.: US 2023/0266299 A1
(43) Pub. Date: Aug. 24, 2023

(54) VEHICLE CONTROL ELEMENT FOR MEASURING CONCENTRATIONS OF SUBSTANCES IN EXHALED AIR AND/OR IN BODY ODOR OF VEHICLE OCCUPANTS

(71) Applicant: ZF Friedrichshafen AG, Friedrichshafen (DE)

(72) Inventors: Johannes Apell, Hergensweiler (DE); Rene Seidemann, Friedrichshafen (DE); Julian King, Rankweil (AT)

(73) Assignee: ZF Friedrichshafen AG, Friedrichshafen (DE)

(21) Appl. No.: 18/171,013

(22) Filed: Feb. 17, 2023

(30) Foreign Application Priority Data
Feb. 18, 2022 (DE) DE102022201701.3

(51) Int. Cl. G01N 33/497; B62D 1/04

(52) U.S. Cl. CPC G01N 33/4972; B62D 1/04; B60K 28/063

(57) ABSTRACT

A vehicle control element measures concentrations of substances in air of vehicle, where the air includes the exhalations and/or body odors or other odors from occupants within the vehicle. A measurement chamber on or in the vehicle control element may include at least one first entry hole through which air can be drawn into the measurement chamber; at least one first exit hole through which air can be discharged from the measurement chamber; a polygonal, elliptical, or circular cross section; reflective inside walls; a laser located at a second entry hole in the measurement chamber, with which laser beams are projected into the measurement chamber through the second entry hole; and a detector located at a second exit hole in the measurement chamber with which laser beams exiting the measurement chamber through the second exit hole are detected. The vehicle control element also includes an interface to an evaluation device.

### RELATED APPLICATION

This application claims the benefit of, and priority to, German Patent Application DE 10 2022 201 701.3, filed Feb. 18, 2022, which is hereby incorporated by reference herein in its entirety.

### TECHNICAL FIELD

The invention relates to a vehicle control element for measuring concentrations of substances in exhaled air and/or in body odors of vehicle occupants, or in other odors in a vehicle.

### BACKGROUND

The following definitions, descriptions and explanations relate and apply to the subject matter of the entirety of the invention disclosed herein.

So-called opacimeters are used for determining the concentration of particles in the exhaust from motor vehicles and other aerosols. These make use of the Beer-Lambert law in that they detect and evaluate the attenuation of light passing through a medium. This is normally involves a light source on one side of a measurement chamber and a light sensor on the other side. Mirror systems are also used in measurement assemblies in order to increase the distance that the light travels. In this case, the light passes through a linear measurement path numerous times. When a very small number of molecules are being measured, as is the case in exhalations, for example, longer measurement paths are necessary. A lengthening of the measurement path by increasing the length of the measurement chamber would result in a larger, impractical measurement device.

DE 10 2011 079 816 A1 discloses a device in this field for measuring amounts of molecules in an aerosol with a measuring chamber in which the aerosol can be placed, a light source that projects a beam of light into the measurement chamber, at least one light sensor for measuring the intensity of the light exiting the measurement chamber, and an evaluation unit for determining the concentration of molecules in the aerosol based on the intensity measured by the sensor. The measurement chamber contains reflective surfaces that reflect the light therein, which are arranged in the shape of a polygon, ellipse, or circle.

DE 10 2014 014 071 A1 discloses a vehicle control element that has a sensor for non-invasive measurement of biomolecules in the blood of a user of the vehicle control element by means of electrochemical impedance spectroscopy.

### BRIEF DESCRIPTION

The object of the invention is be able to miniaturize a laser spectroscopy measurement chamber for analysis of odor gas/exhalation gas in a vehicle, such that it can be integrated in a confined space.

In particular, the invention results in a vehicle operating element for measuring concentrations of substances in exhalations and/or body odors of vehicle occupants. The vehicle control element comprises at least one measurement chamber placed on or in the vehicle control element. The measurement chamber comprises at least one first entry hole through which the exhalations and/or body odors can be drawn into the measurement chamber, and at least one exit hole through which the exhalations and/or body odors can be discharged from the measurement chamber. The measurement chamber also comprises a polygonal, elliptical, or circular cross section. The inner surfaces of the measurement chamber are reflective. The measurement chamber also comprises a laser located at a second entry hole in the measurement chamber, with which laser beams are projected through the second entry hole into the measurement chamber, and a detector located at a second exit hole in the measurement chamber with which the laser beams exiting the second exit hole in the measurement chamber are detected. The measurement chamber also comprises an interface for an evaluation device that determines the concentrations of substances in the exhalations and/or body odors from the detected laser beam on the basis of a laser spectroscopic method.

A vehicle occupant controls functions of a vehicle using a vehicle control element. The vehicle control element can be a control element for a vehicle HVAC system, e.g. a touchscreen, or a push button, or a knob. The vehicle control element can also be a control element for an infotainment system, e.g. a touchscreen or button or knob. According to one aspect of the invention, the vehicle control element is a control element with which the distance between vehicle occupants and the vehicle control element can be reduced when used, e.g. by the vehicle occupants leaning forward, toward the vehicle control element. According to one aspect of the invention, the vehicle control element is used for steering the vehicle, e.g. a steering wheel or a driving and/or steering element for handicapped operators, e.g. a multifunctional knob placed on a steering wheel.

Body odors comprise all detectable human scents emitted through the skin or other bodily orifices, e.g. breath odors. Substances in exhalations and/or body odors can be substances indicating alcohol, cocaine, amphetamines, cannabis, tetrahydrocannabinol, morphine, methadone, ammonia, acetone, or a combination thereof. These substances represent biomarkers that can indicate a normal biological or abnormal process in the body. By way of example, the smell of ammonia indicates kidney disease. The smell of acetone indicates diabetes. One advantage in detecting these substances in the exhalations and/or body odors is that there is no need for chemical methods used to determine alcohol content, requiring the unpleasant process of blowing into a breathalyzer, or surface contact methods, e.g. adsorption methods, or invasive methods. Exhalations and other body odors are continuously emitted. The substances are volatile organic compounds, for example, also abbreviated as VOCs. VOCs include, e.g., acetone, ethanol, isoprene, nonanal, decanal, a-pinene, ethyl butyrate and butanal, ethanal, propionaldehyde, and propyl acetate. VOCs occur in animals in the course of protein, cellular, or metabolic changes triggered by a disease. By way of example, characteristic protein and metabolic changes have been observed in humans infected with SARS-Cov-2. VOCs emitted through exhalation and/or body odor can be characteristic for a disease, thus functioning as a biomarker.

The detection of these substances with the invention is used to determine the state of a vehicle occupant, in particular a vehicle operator, with regard to illness and driving incapacity. If certain substances exceed a respective predefined threshold value, in particular when starting travel, a vehicle immobilizer is activated according to one aspect of the invention, e.g. an alcolock or breath alcohol ignition interlock device, which blocks the vehicle's ignition if the vehicle operator's breath alcohol exceeds a specific value.

The first entry and exit holes enable a homogenous distribution of a volume of the exhalations and/or body odors that are to be currently analyzed in the measurement chamber.

The laser beams are reflected multiple times on the reflective inner surfaces of the walls of the measurement chamber. This results in a travel distance within the measurement chamber that is greater than the diameter of thereof. Consequently, a miniaturized measurement chamber is obtained for the analysis of odors/exhalations in a vehicle, that can be installed in the interior thereof. This results in a measurement chamber that is extremely effective, despite its small size, and is therefore ideal for measuring substances in exhalations and/or body odors in vehicles.

By arranging the reflection surfaces in the shape of a polygon, it is possible to obtain a specific reflection angle.

The detector can be a photodiode detector.

The evaluation device can comprise application-specific integrated circuits, ASICs, field-programmable gate arrays, FPGAs, central processing units, CPUs, or graphics processing units, GPUs. The evaluation device can be a microcontroller or a control unit. The control unit can be an electric control unit.

Laser spectroscopic methods comprise absorption spectroscopy and emission spectroscopy.

According to one aspect of the invention, the first entry hole and/or the first exit hole contain a filter for filtering exhalations and/or body odors, and/or a valve for regulating and/or controlling the flow of the exhalations and/or body odors through the measurement chamber. The filter is used to improve the signal/noise ratio, for example. The valve improves the distribution of the exhalations and/or body odors that are to be analyzed in the measurement chamber. According to one aspect of the invention, the first entry hole contains a ventilator for suctioning in the exhalations and/or body odors.

According to another aspect of the invention, the inner surfaces of the walls of the measurement chamber comprise reflectors that can be heated, e.g. mirrors that can be heated. This prevents condensation from forming inside the measurement chamber. The reflectors are heated to a temperature of at least 19° C., for example.

According to another aspect of the invention, the vehicle control element comprises at least one vibrator for cleaning the inner surfaces, in particular for removing particles adhering to the reflectors. The vibrator can be formed by an ultrasonic source, for example.

According to another aspect of the invention, the evaluation device is integrated in the detector or in the vehicle control device. By way of example, the detector is a system on a chip, or SoC.

According to another aspect of the invention, the evaluation device determines a spectrum, e.g. an absorption or emission spectrum. The evaluation device also executes a computer program. The computer program comprises commands with which a machine learning model trained for image classification derives the concentration of the substances in the exhalations and/or body odors on the spectrum.

The commands executed by the evaluation device are program instructions or hardware commands. The commands are in the form of software code or machine code. Machine learning is a technology learned by computers and other data processing devices for executing tasks by learning from data, instead of being programmed for the tasks.

According to another aspect of the invention, the vehicle control element is a steering wheel. The measurement chamber is located on a steering wheel rim, steering wheel spoke, or steering column. This makes it easy for the driver to use because the distance between the driver and steering wheel can be reduced to less than 20 cm by leaning forward, which has advantages with regard to detecting substances in the exhalations and/or body odors. The steering wheel is a multi-function steering wheel, by way of example.

According to another aspect of the invention, the measurement chamber is formed in the horn button on a steering wheel, or in a mount for the horn button. In particular in the embodiment in which the evaluation device is integrated in the detector, a horn button or mount for the horn button is obtained with which the concentration of substances in exhalations and/or body odors of vehicle occupants can be measured.

### BRIEF DESCRIPTION OF THE DRAWINGS

Exemplary embodiments of the invention are illustrated in the drawings.

### Therein:

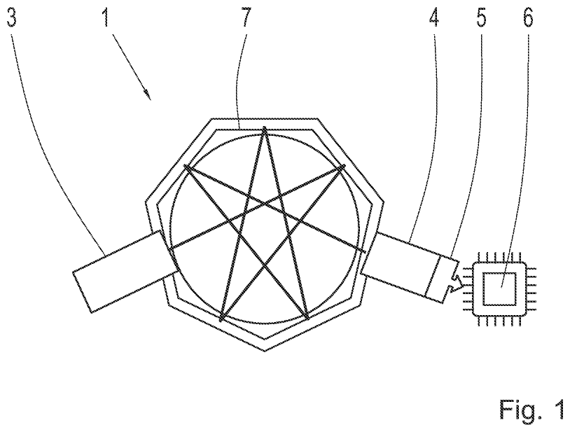

FIG. 1 shows an exemplary embodiment of a measurement chamber according to the invention;

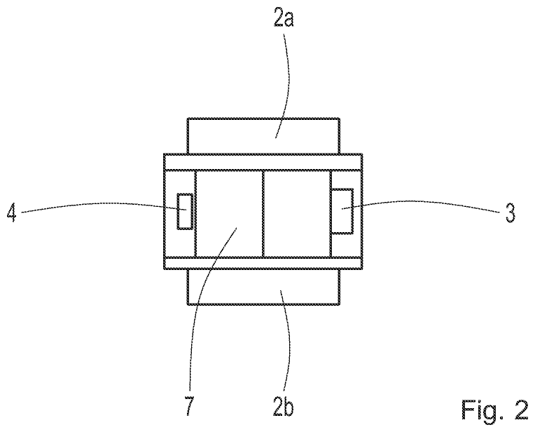

FIG. 2 shows a side view of the exemplary embodiment shown in FIG. 1;

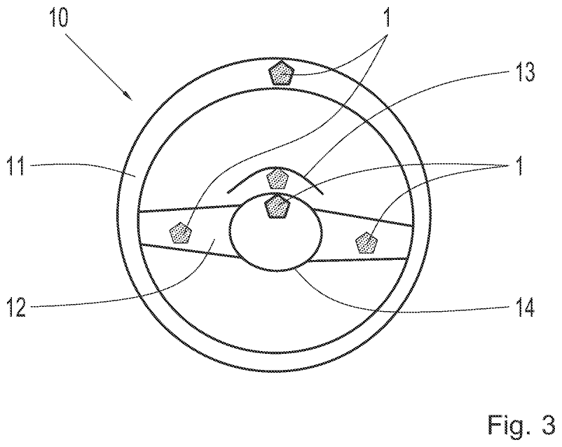

FIG. 3 shows an exemplary embodiment of a vehicle control element according to the invention;

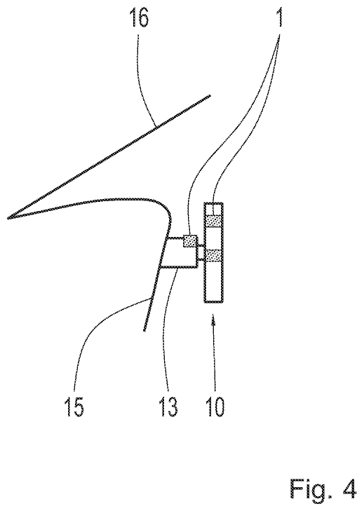

FIG. 4 shows a side view of the exemplary embodiment shown in FIG. 3; and

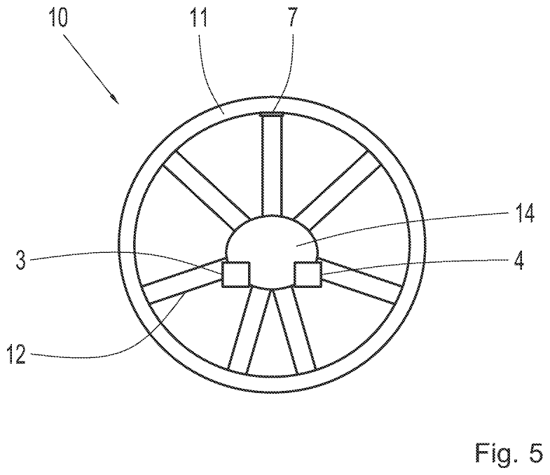

FIG. 5 shows another exemplary embodiment of a vehicle control element according to the invention.

The same reference symbols are used for identical or functionally similar elements in the drawings.

### DETAILED DESCRIPTION OF THE DRAWINGS

The measurement chamber 1 in FIG. 1 has a heptagonal cross section, by way of example. The laser 3 and detector 4 are placed on the measurement chamber 1. The signals from the detector 4 are sent to the evaluation unit 6 via the interface 5. The detector 4, interface 5, and evaluation device 6 can be in the form of a system on a chip, or SoC. Reflectors 7 in the form of mirrors are located on the inner surfaces of the walls of the measurement chamber 1.

In another exemplary embodiment of the measurement chamber 1 according to the invention, the laser 3 is tilted slightly. The detector 4 is lower than the laser 3. This results in the laser beam circulating through the measurement chamber 1 more than once, thus lengthening the path of the laser beam within the measurement chamber 1.

The first entry hole 2a and first exit hole 2b are shown in FIG. 2 in the side view of the measurement chamber from FIG. 1.

FIG. 3 shows the possible positions where the measurement chamber 1 can be installed in the vehicle control element 10, e.g. a steering wheel. One or more measurement chambers 1 can be placed in a steering wheel rim 11. One or more measurement chambers 1 can be placed in steering wheel spokes 12. One or more measurement chambers 1 can be placed in a steering column 13. One or more measurement chambers 1 can be place in a horn button 14, or on or in a mount for the horn button 14. By way of example, a first measurement chamber 1 is located in the steering wheel rim 11, a second and third measurement chamber 1 are located in the steering wheel spokes 12, a fourth measurement chamber 1 is located in the steering column 13, and a fifth measurement chamber 1 is located in the horn button 14.

FIG. 4 shows a side view of FIG. 3 and illustrates where the measurement chamber 1 is installed. The steering column 13 connects the steering wheel to a dashboard 15 and forms the connection to the steering column assembly. The steering column 13 comprises a steering column switch, e.g. a lever for turning signals, high beam flasher, windshield wipers, and/or cruise control. The front windshield 16 is also shown.

FIG. 5 shows the horn button 14, which forms the measurement chamber 1.

### REFERENCE SYMBOLS

1 MEASUREMENT CHAMBER
2a first entry hole
2b first exit hole
3 laser
4 detector
5 interface
6 evaluation device
7 reflector
10 vehicle control element
11 steering wheel rim
12 steering wheel spoke
13 steering column
14 horn button
15 dashboard
16 windshield

1. A vehicle control element for measuring concentrations of substances the air within a vehicle, comprising:
an interface to an evaluation device with which the concentration of the substances is determined from a laser beam on the basis of a laser spectroscopic method; and
at least one measurement chamber, in which the measurement chamber comprises:
at least one first entry hole through which the air is drawn into the measurement chamber;
at least one first exit hole through which the air is discharged from the measurement chamber;
at least one of a polygonal, elliptical, or circular cross section;
reflective inside walls;
a laser located at a second entry hole in the measurement chamber, with which the laser beam is projected into the measurement chamber through the second entry hole;
a detector located at a second exit hole in the measurement chamber with which the laser beam exiting the measurement chamber through the second exit hole are detected.

2. The vehicle control element according to claim 1, wherein the first entry hole and/or the first exit hole include a filter for filtering the air.

3. The vehicle control element according to claim 1, wherein the first entry hole and/or the first exit hole include a valve for controlling a flow of the air through the measurement chamber.

4. The vehicle control element according to claim 1, wherein the inner surfaces of the walls of the measurement chamber comprise reflectors that are configured for heating.

5. The vehicle control element according to claim 1, comprising at least one vibrator for cleaning the inner surfaces.

6. The vehicle control element according to claim 1, wherein the evaluation device is integrated in the detector or in the vehicle control element.

7. The vehicle control element according to claim 5, wherein the evaluation device determines a spectrum and executes a computer program that contains commands with which a machine learning model trained for image classification derives the concentration of substances in the air on the spectrum.

8. The vehicle control element according to claim 1, wherein the vehicle control element is a steering wheel and the measurement chamber is located on a steering wheel rim, on a steering wheel spoke, or on a steering column.

9. The vehicle control element according to claim 1, wherein a horn button on a steering wheel or a mount for the horn button forms the measurement chamber.

10. The vehicle control element according to claim 1, wherein the air includes exhalations and/or body odors from passengers within the vehicle, and wherein the vehicle control element is configured to measure the concentrations of the substances sourced from the exhalations and/or body odors.
